(19) United States
(12) Patent Application Publication
(10) Pub. No.: US 2025/0127539 A1
(43) Pub. Date: Apr. 24, 2025

(54) Device for the Placement of Organic Tissue and Relative Apparatus

(71) Applicants: IRCCS AZIENDA OSPEDALIERO - UNIVERSITARIA DI BOLOGNA, Bologna (IT); ALMA MATER STUDIORUM - UNIVERSITA' DI BOLOGNA, Bologna (IT)

(72) Inventors: Paolo Casadio, Bologna (IT); Renato Seracchioli, Bologna (IT); Diego Raimondo, Bologna (IT); Benedetta Orsini, Bologna (IT); Alessandro Arena, Bologna (IT); Antonio Raffone, Bologna (IT)

(73) Assignees: IRCCS AZIENDA OSPEDALIERO - UNIVERSITARIA DI BOLOGNA, Bologna (IT); ALMA MATER STUDIORUM - UNIVERSITA' DI BOLOGNA, Bologna (IT)

(21) Appl. No.: 18/690,535

(22) PCT Filed: Sep. 7, 2022
(86) PCT No.: PCT/IB2022/058418

§ 371 Date: Mar. 8, 2024

(30) Foreign Application Priority Data
Sep. 9, 2021 (IT) 102021000023285

(51) Int. Cl. A61B 17/34; A61B 17/00; A61B 17/425

(52) U.S. Cl. CPC A61B 17/3468; A61B 17/425; A61B 2017/00969

(57) ABSTRACT

A device for the placement of organic tissue, in particular endocrine tissue, includes a hollow tubular base element configured to house in a seat at least one portion of the organic tissue, a pushing element which inserted on the inside of the base element and coupled to it in a sliding manner; wherein the base element is configured to at least partially penetrate a target tissue. The base element further includes a first opening, which is arranged in a distal zone towards a first end and is configured to let out and, hence, position the organic tissue in the target tissue through the action of the pushing element.

### CROSS-REFERENCE TO RELATED APPLICATIONS

This patent application claims priority of Italian Patent Application No. 102021000023285 filed on Sep. 9, 2021, the entire disclosure of which is incorporated herein by reference.

### FIELD OF THE ART

The present invention relates to a device for the placement of organic tissue and a relative apparatus.

In particular, the present invention finds advantageous, but not exclusive application in the orthotopic or heterotopic placement of endocrine tissue, in particular ovarian, to which the following description will make explicit reference without thereby losing generality.

### BACKGROUND OF THE INVENTION

In the field of reproductive medicine for the preservation of fertility and ovarian function, techniques are known for reimplantation of ovarian tissue in patients undergoing, for example, oncological therapy, which determines, in multiple cases, at least a partial suppression of the ovarian function, which is a potential cause of infertility and/or premature menopause (pathologies that are known in themselves and therefore not further detailed here).

For some years, patients waiting to undergo oncological therapy with a risk of suppression of the ovarian function are usually subjected to a preventive biopsy of ovarian tissue, which is preserved by cryopreservation. At the end of chemo-and/or radiotherapy therapy, patients may undergo reimplantation of the previously harvested tissue, with subsequent restoration of the hormonal and gametogenic (particularly ovogenic) function.

In detail, considering the increase in life expectancy, it is important to guarantee to the aforesaid type of patients an improvement in the quality of life, with particular attention to the field of oncofertility, i.e. that area of interest of the reproductive medicine that aims at safeguarding the fertility of patients undergoing cancer treatments. In particular, ovarian tissue cryopreservation plays an important role in this context and is currently the main mode of preservation of hormonal function and fertility in this type of patients.

In detail, according to the prior art, at the end of the oncological therapy, the previously harvested ovarian tissue is reimplanted either orthotopically (i.e. in the area of the retroperitoneal pelvic space) by endoscopy or heterotopically (e.g. in the subcutaneous tissue) by transcutaneous incisional route.

Therefore, both in the case of a transcutaneous incisional approach, and in the case of a laparotomic/endoscopic approach (e.g. laparoscopic), analgesia/anaesthesia techniques must be used (with the known relative risks, possible hospitalisation and costs for the patient and/or for the healthcare system).

Moreover, the method according to the known art presents non-negligible surgical risks, which often also cause a strong psychological impact on the patient (e.g. due to the surgery itself and/or the consequent aesthetic result that may result).

Aim of the present invention is to realise a device for the placement of organic tissue and relative apparatus, which are at least partially free of the drawbacks described above and, at the same time, are simple and inexpensive to manufacture.

### SUMMARY

In accordance with the present invention, there are provided a device for the placement of organic tissue and a relative apparatus as claimed in the following independent claims and, preferably, in any one of the claims directly or indirectly dependent on the independent claims.

The claims describe preferred embodiments of the present invention forming an integral part of the present disclosure.

### BRIEF DESCRIPTION OF THE DRAWINGS

The present invention will now be described with showing some non-reference to the enclosed drawings, limiting embodiments thereof, wherein:

FIG. 1 shows schematically and with details removed for clarity's sake, a first variant of a device in accordance with the present invention and for percutaneous use;

FIG. 2 shows, schematically and with details removed for clarity's sake, a second variant of a device in accordance with the present invention and for endoscopic use;

FIGS. 3a-3e show, schematically and with details removed for clarity's sake, a side sectional view of a first embodiment of a device in accordance with the present invention in different operating configurations;

FIG. 3f shows, schematically and with details removed for clarity's sake, a view from below of the embodiment of FIGS. 3a-3e;

FIGS. 4a-4d show, schematically and with details removed for clarity's sake, a side sectional view of a second embodiment of a device in accordance with the present invention in different operating configurations;

FIGS. 5a-5d show, schematically and with details removed for clarity's sake, a side sectional view of a third embodiment of a device in accordance with the present invention in different operating configurations;

FIGS. 6a-6d show, schematically and with details removed for clarity's sake, a side sectional view of a fourth embodiment of a device in accordance with the present invention in different operating configurations;

FIGS. 7a-7c show, schematically and with details removed for clarity's sake, a side sectional view of a fifth embodiment of a device in accordance with the present invention in different operating configurations;

FIGS. 8a-8e show, schematically and with details removed for clarity's sake, a side sectional view of a sixth embodiment of a device in accordance with the present invention in different operating configurations;

FIGS. 9a-9d show, schematically and with details removed for clarity's sake, a side sectional view of a seventh embodiment of a device in accordance with the present invention in different operating configurations; and

FIGS. 10a-10d show, schematically and with details removed for clarity's sake, a side sectional view of an eighth embodiment of a device in accordance with the present invention in different operational configurations.

### DETAILED DESCRIPTION

With reference to the accompanying Figures, number 1 denotes a device for the placement of an organic tissue 2.

In particular, the organic tissue 2 is endocrine tissue, i.e. belonging to glands/organs suitable for the production of hormones. For example, the organic tissue 2 is ovarian, pancreatic, or thyroid tissue.

In the following description, the same numbers and the same reference letters in the figures identify the same elements or components with the same function.

In the context of this disclosure, the term “second” component does not imply the presence of a “first” component. These terms are in fact used as labels to improve clarity and should not be understood in a limiting way.

The elements and features shown in the various preferred embodiments, including the drawings, may be combined with each other without thereby departing from the scope of protection of the present application as described below.

The device 1 comprises a hollow tubular base element 3, which determines, on the inside thereof, a primary duct 4 configured to house in a seat 5 at least one portion of the organic tissue 2 to be positioned/implanted in a patient P. The base element 3 comprises an end 6 and an end 7 opposite the end 6 relative to the primary duct.

Advantageously, the base element 3 is configured to at least partially penetrate a target tissue 8.

Preferably but not in a limiting way, the end 6 of the base element 3 comprises a cutting profile for at least partially (in particular completely and therefore crossing) pierce a tissue that covers the target tissue 8.

Preferably but not in a limiting way, the end 6 of the base element 3 comprises a cutting profile for at least partially piercing the target tissue 8.

In particular, the base element is a needle element, that is, with the tip (at the end 6) cut obliquely to obtain a cutting profile capable of piercing the tissues of a patient P (with minimum trauma) to reach the site where the organic tissue 2 to be positioned/implanted in a patient P is to be deposited. In this way, it is possible to avoid having to cut the tissues (for example cutaneous ones) that cover the target tissue 8 (and part of the target tissue 8 itself) in advance to be able to deposit the organic tissue 2 in the desired position, thus reducing the risks of infection and/or imperfections for the patient P.

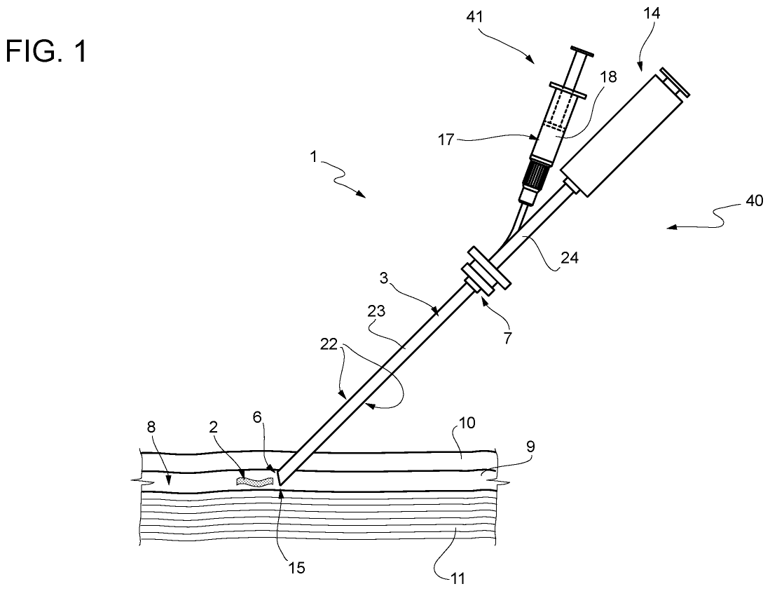

According to the non-limiting embodiment of FIG. 1, the device 1 is configured for percutaneous use, in which the target tissue 8 is a subcutaneous tissue 9, which comprises (is) a space between a portion of skin 10 and of muscle tissue 11 (in particular between skin 10 and muscle aponeurosis).

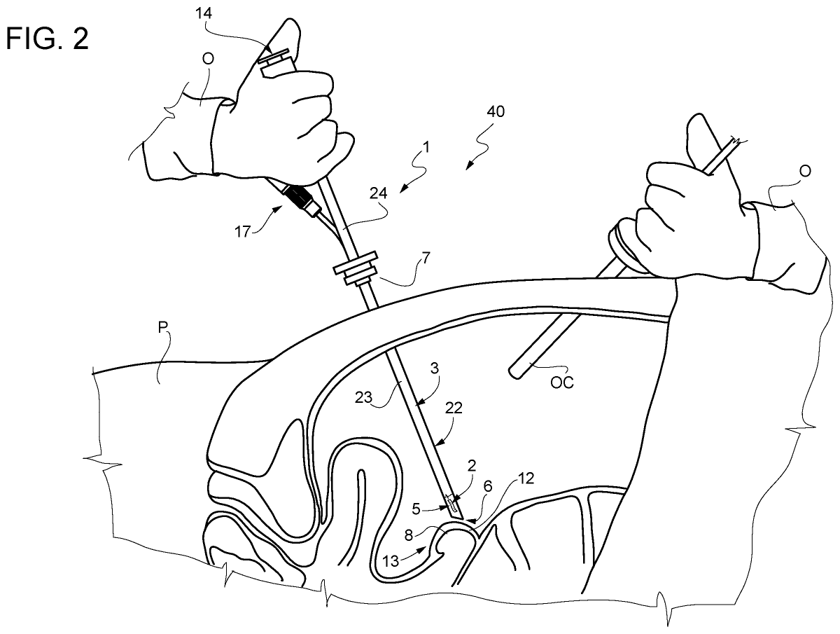

In the non-limiting embodiment of FIG. 2, however, the device 1 is configured for endoscopic use, wherein the target tissue 8 is preferably below a portion of peritoneum 12, in particular in the area of the retroperitoneal pelvic space 13 (more particularly in the area of an ovarian fossa).

In particular, the device 1 is controlled by an operator O, who performs the surgery with the aid of the OC optics of a video camera (known in itself and therefore not further detailed) (or without, in the case of percutaneous placement).

Obviously, the dimensions of the device 1 for percutaneous use and of the device 1 for endoscopic use can be different and in particular vary in length and diameter in light of the different dimensions allowed by the two modes of use. In particular, the device 1 for percutaneous use has at least one dimension which is smaller than the device 1 for endoscopic use.

In some non-limiting cases, the base element 3 has a substantially circular cross-section, in other non-limiting cases, in order to increase the dimensions (width) of the organic tissue 2 to be positioned (usually supplied as strips), the base element 3 has an oval cross-section.

Advantageously, the device 1 further comprises a pushing element 14, which can be inserted or is inserted on the inside of the base element 3 (in particular from the end 7) and is coupled to it in a sliding manner along a longitudinal axis A.

Furthermore, the device 1 comprises an opening 15 which is arranged in a distal zone DZ towards the end 6. The opening 15 is configured to let out and, hence, position the organic tissue 2 in the target tissue 8 through the action of the pushing element 14.

In particular, the pushing element 14 is operable by an operator O (e.g., a surgeon) to position the organic tissue 2 once the opening 15 has reached the desired point in the target tissue 8.

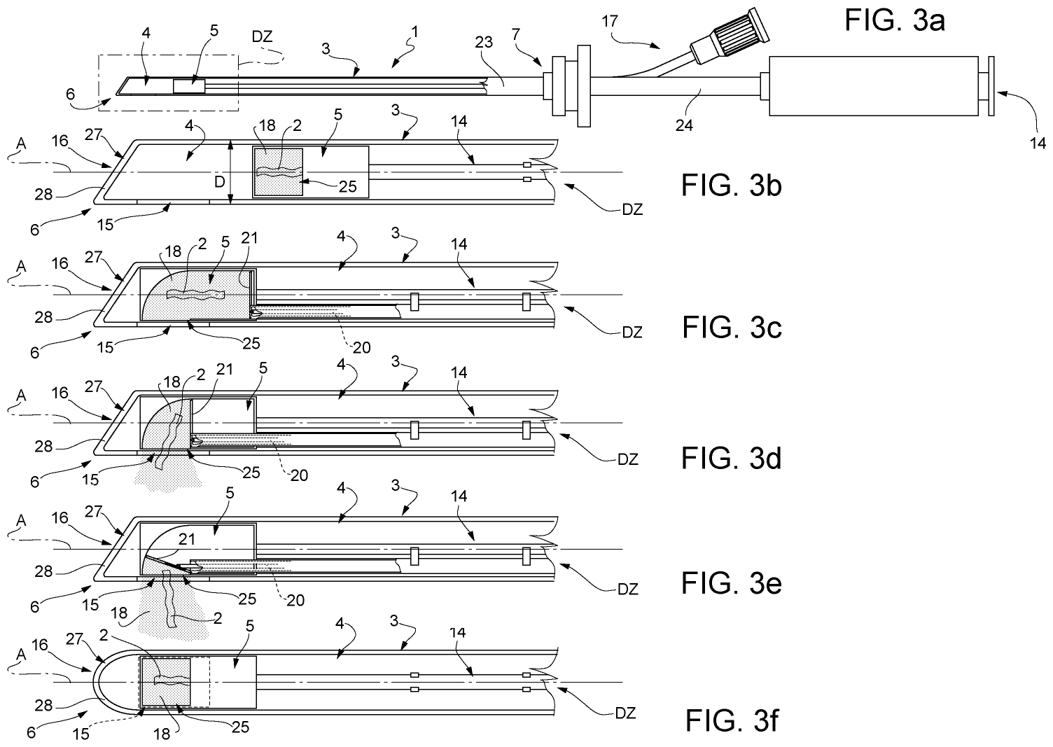

Preferably, the device 1 comprises a protection element 16 for the organic tissue 2 which is configured to protect the organic tissue 2 while the device 1 (i.e. the base element 3) penetrates towards the target tissue 8. In particular, the protection element 16 is configured to assume at least one closed configuration (shown for example in FIGS. 3a, 3b, 4a, 4b, 5a, 5b, 6a, 7a, 8a, 8b, 9a, 10a and 10b) in which it at least partially, in particular totally, closes the opening 15, and (preferably) an open configuration (shown for example in FIGS. 3c-f, 4c-d, 5c-d, 6c-d, 7b-c, 8c-e, 9c-d, 10c-d), in which the protection element 16 allows the organic tissue 2 to come out, and hence, be positioned in the target tissue 8. In this way, it is possible to prevent the organic tissue 2 from being damaged or its correct placement from being thwarted during the penetration towards the target tissue 8, preventing the entry of fat and/or other soft tissue into the primary duct 4.

Advantageously but not necessarily, the device 1 comprises at least one injection device 17 which is configured to inject into the target tissue 8 a culture liquid 18 (of known composition and therefore not more detailed below), which is configured to enhance the activity and/or the reproduction of the organic tissue 2 in the target tissue 8, enhancing implantation.

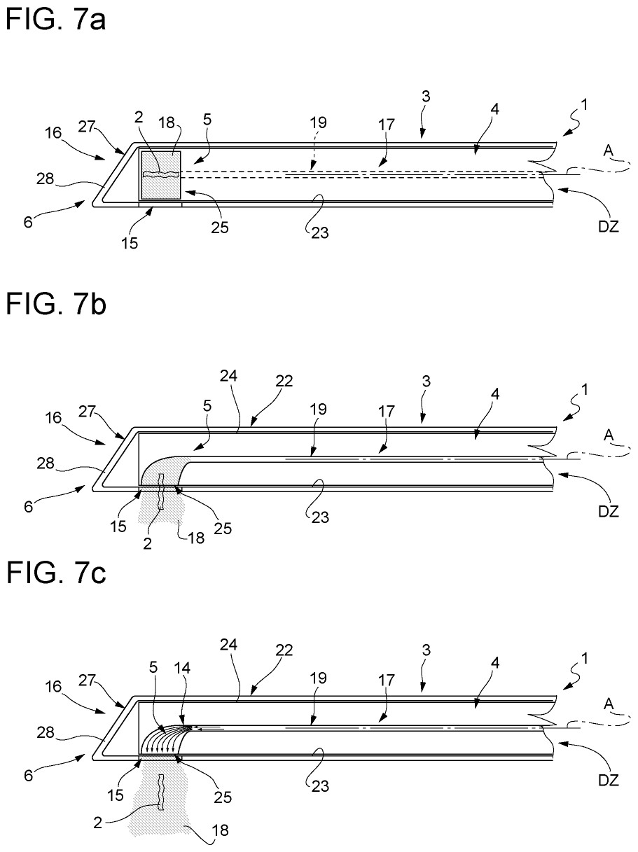

In particular, the injection device 17 comprises a channel 19 on the inside of the primary duct 4 through which the culture liquid 18 flows. In some non-limiting cases, the inner channel 19 is defined by the primary duct 4 itself. In other non-limiting cases, such as those shown in FIGS. 7a-c and 10a-d, the inner channel 19 is independent of the primary duct 4, in particular it has a diameter which is smaller than the primary duct 19 and which is preferably arranged concentrically to the primary duct 19 itself. In this way, the desired pressures to push the organic tissue 2 through the opening 15 can be achieved.

Advantageously but not necessarily, the injection device 17 is configured to be operated before and/or after and/or during the placement of the organic tissue 2. In this way, a suitable housing can be created inside the target tissue 8 to accommodate the organic tissue 2.

According to some non-limiting embodiments, for example those shown in FIGS. 3a-6d and 8a-9d, the pushing element 14 comprises a pushing member 20 provided with a piston 21, which at least partially longitudinally (i.e. along the axis A) goes through the base element 3 and is configured to push the organic tissue 2 through the opening 15 following a relative movement between the piston 21 and the base element 3. In other words, the pushing member 20 is a mechanical element operable by the operator O to impart a force on the tissue 2 and push it out of the base element 3, through the opening 15, into the target tissue 8.

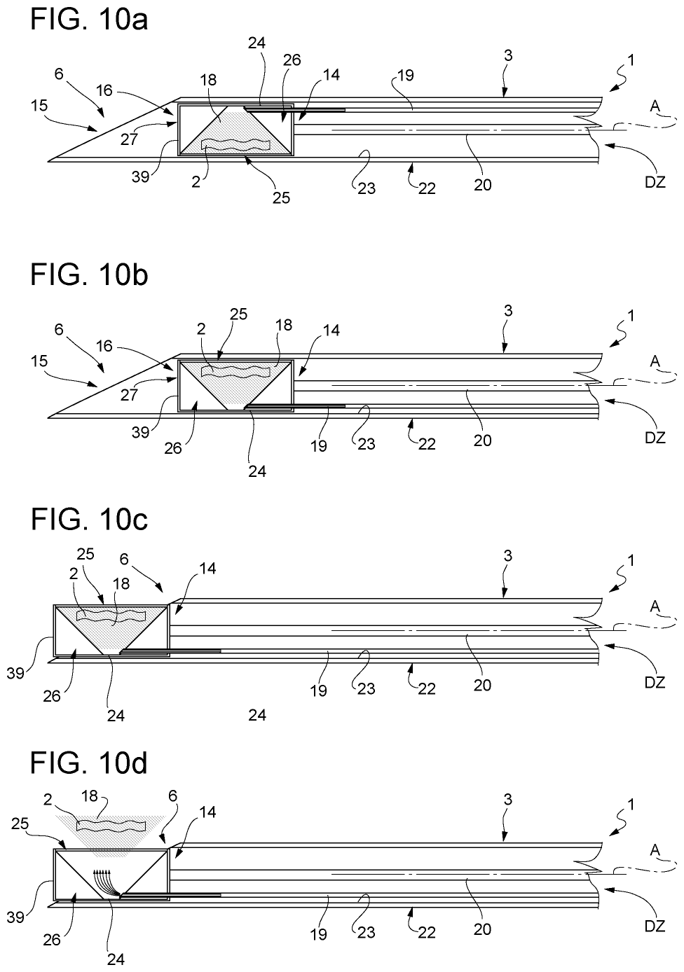

According to other non-limiting embodiments, for example those shown in FIGS. 7a-7c and 10a-10d, the pushing element 14 is defined by the culture liquid 18, which at least in part longitudinally (i.e. along the axis A) flows through the primary duct 4 (in particular through the inner channel 19) and is configured to push (hydraulically) the organic tissue 2 through the opening 15 following a relative movement between the culture liquid 18 and the base element 3. In other words, the pushing function above performed by the pushing member is in this case carried out hydraulically by the culture fluid 18, which, subjected to a pressure imparted by the injection device 17, imparts a force to the tissue 2 driving it out of the base element 3, through the opening 15, into the target tissue 8. In this way, the device 1 is mechanically simple and at the same time facilitates the function of the organic tissue 2 through the culture liquid 18.

According to further non-limiting and non-illustrative embodiments, the pushing element 14 comprises both a pushing member 20 and the culture liquid 18 as described.

In some non-limiting cases, such as those shown in FIGS. 3a-3f and 7a-7c, the opening 15 is arranged along a side wall 22 of the base element 3 so as to let out the organic tissue 2 crosswise to a longitudinal axis A of the base element 3. In particular, the opening 15 is parallel to the longitudinal axis A.

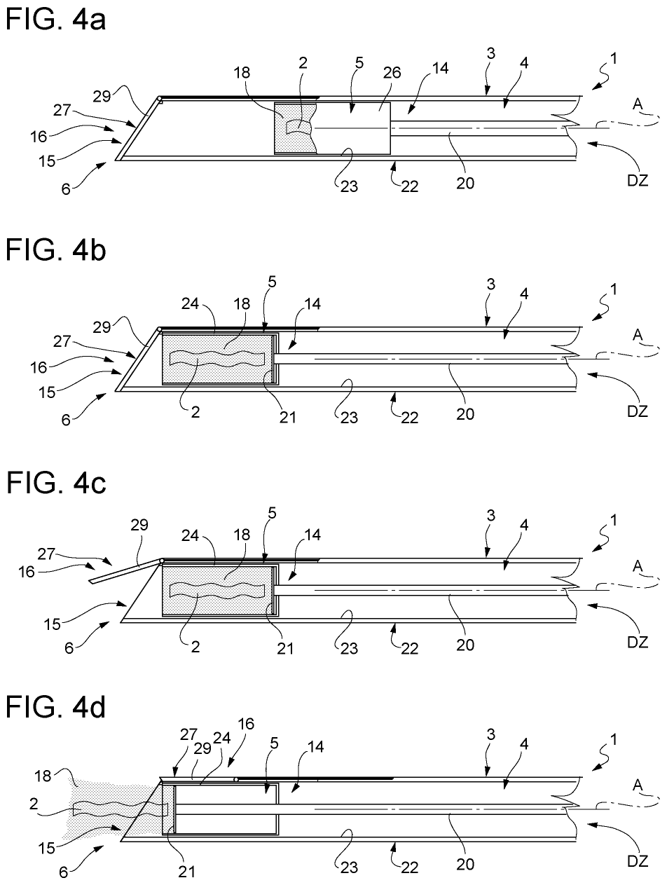

Alternatively (FIGS. 4a-6d and 8a-9d) or additionally (FIGS. 10a-10d), the opening 15 is arranged in the area of the end 6 so as to let out the organic tissue 2 crosswise (FIGS. 10a-10d) or parallel (FIGS. 4a-6d and 8a-9d) to the longitudinal axis A of the base element 3. In particular, the opening 15 is defined as continuation of the primary duct 4. In other words, the opening 15 is the tip of the needle.

In some non-limiting and not shown cases, the organic tissue 2 is divided into different portions that are placed through the same opening 15 or through different openings 15 arranged in the area of the distal zone DZ.

According to some non-limiting embodiments, the base element 3 defines an outer shell 23. In particular, the protection element 16 of the organic tissue 2 is defined at least partially by a hollow tubular inner shell 24 which is arranged inside the outer shell 23 in a concentric manner and is configured to house the seat 5 (thus the portion of the organic tissue 2 to be positioned) and the pushing element 14. More precisely, the inner shell 24 comprises an opening 25 which is available in the distal zone DZ (adjustable towards the opening 15) and configured to let out the organic tissue 2 towards the opening 15.

In particular, the inner shell 24 is at least capable of translating along and/or of rotating around the longitudinal axis A so as to cause the opening 15 and the opening 25 to become complementary to (i.e. facing) one another, thus determining a connection between the inside of the inner shell 24 and the outside of the outer shell 23 in the target tissue 8. In other words, preferably, in such embodiments the shells 23 and 24 are spaced from each other, that is, provided with two degrees of freedom, one of which is linear (they are free to slide mutually along the longitudinal axis A) and one is rotatable (they are free to rotate mutually around the longitudinal axis A).

Advantageously but not necessarily, the inner shell 24 can be inserted on the inside of the outer shell 23 from the end 7.

According to some non-limiting embodiments, the shell 24 comprises a cup-shaped body 26, which is configured to contain the organic material 2 and/or the culture liquid 18. In this way, before inserting the shell 24 inside the shell 23, the operator O fills the cup-shaped body 26, which in this case defines the seat 5, with the organic material 2 surrounded by the culture liquid 18.

Advantageously but not necessarily, the primary duct has a diameter D which is smaller than or equal to 3 mm (i.e. with gauge greater than or equal to 8), in particular smaller than 2 mm (i.e. with gauge greater than or equal to 12), more particularly, especially in the case of percutaneous use, lower than 1.5 mm (i.e. with gauge greater than or equal to 14), in detail lower than 1 mm (i.e. with gauge greater than or equal to 18).

Advantageously but not necessarily, the base element 3 has a diameter which is smaller than 5 mm, in particular smaller than 3 mm, more particularly, especially in the case of percutaneous use, smaller than 2 mm, in detail smaller than 1.5 mm.

Advantageously but not necessarily, and as shown in the non-limiting embodiments of the accompanying figures, the protection element 16 comprises an obstructing body 27.

In the non-limiting embodiment of FIGS. 3a-f and 7a-c, the obstructing body 27 is defined by an end wall 28 of the base element 3. In particular, the end wall 28 is oblique with respect to the longitudinal axis A, so as to allow in any case the device to penetrate into the target tissue 8, thus maintaining a sharp profile.

In the non-limiting embodiment of FIGS. 4a-d, the obstructing body 27 is defined by a retractable element 29 which is initially arranged in the area of the opening 15 and is configured to close the primary duct 4 during the penetration towards the target tissue 8 and to subsequently be retracted into a respective seat 30 which is preferably obtained in the area of the side wall 22 of the base element 3.

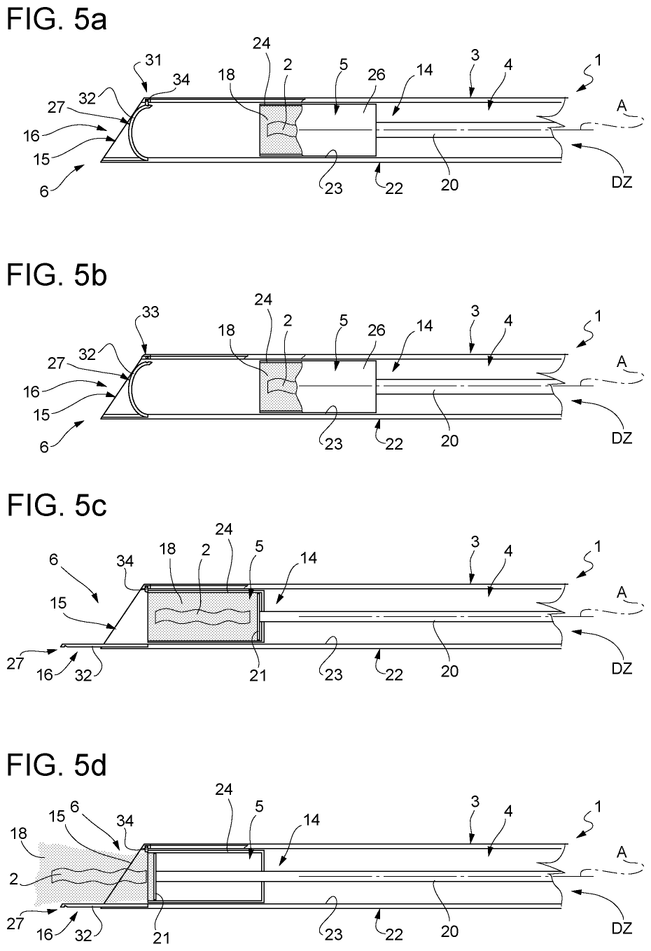

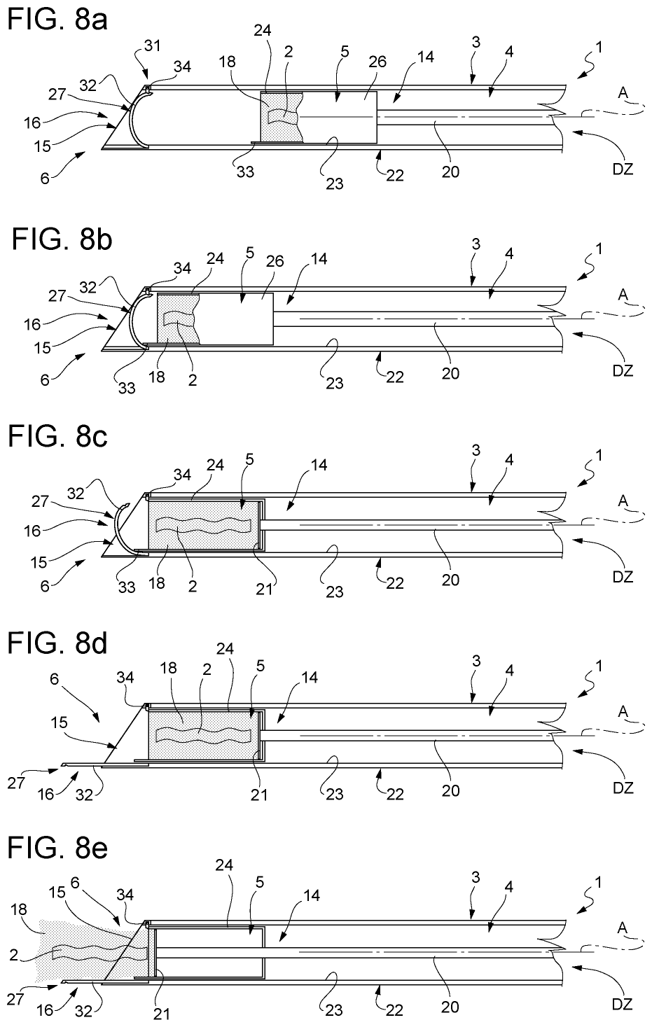

In the non-limiting embodiment of FIGS. 5a-5d and 8a-e, the obstructing body 27 is defined by a mechanical opening device 31 (comprising an elastic body 32) which is arranged in the area of the opening 15 and is configured to close the primary duct 4 during the penetration towards the target tissue 8 and to free the opening 15 by means of a release system 33. In the non-limiting embodiment of FIGS. 5a-5d, the release system 33 is defined by a pin 34, which is retracted as a function of a command of the operator O, allowing the elastic body 32 to return to shape (FIGS. 5c and 5d) and the organic tissue 2 to come out for operating the pushing element 14. In the non-limiting embodiment of FIGS. 8a-e, the release system 33 is defined by the pushing element 14, which, by pushing against the elastic body 32, releases it from the (fixed) pin 34 and allows the elastic body 32 to return to shape (FIGS. 8c-e) and the organic tissue 2 to come out for operating the pushing element 14.

Advantageously but not necessarily, the pin 33 also acts as an end stop for the cup-shaped body 26 and/or for the piston 21.

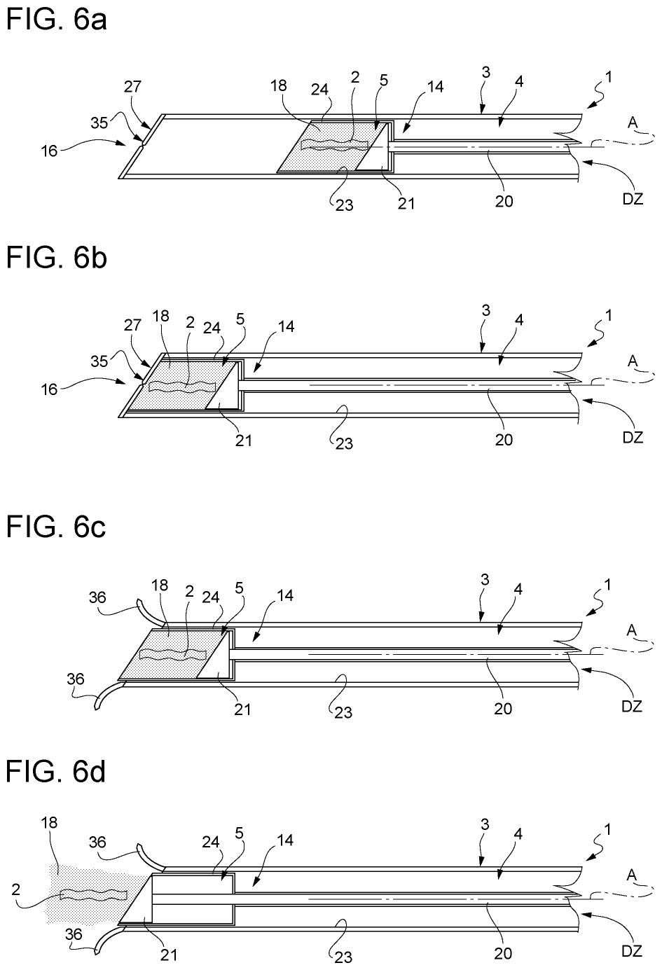

In the non-limiting embodiment of FIGS. 6a-d, the obstructing body 27 is defined by a membrane 35 which preferably is pre-cut and is configured to be opened by the pushing element 14. Preferably but not necessarily, the membrane 35 has one or more V-shaped pre-cuts to facilitate the separation thereof into flaps 36.

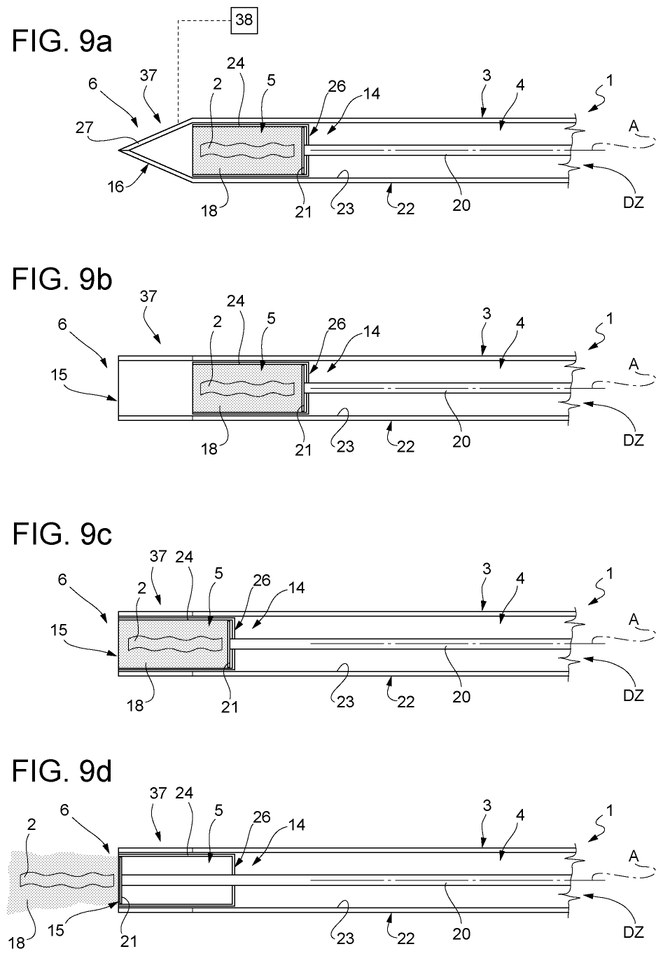

In the non-limiting embodiment of FIGS. 9a-d, the obstructing body 27 is defined by a portion 37 of the base element 3 in the area of the distal zone DZ made of a (alloy) shape memory material (Shape Memory Alloy) which is controlled by a control system 38 (in particular thermal or thermoelectric) so as to shift from the closed configuration (FIG. 9a, maintained during the penetration of the target tissue) to the open configuration (FIGS. 9b-d, for the placement of organic tissue 2).

In the non-limiting embodiment of FIGS. 10a-d, the obstructing body 27 is defined by a portion 39 of the cup-shaped body 26 defining the seat 5.

Advantageously but not necessarily, the seat 5 can be loaded by the operator O with the organic tissue 2 and/or the liquid 18 immediately before proceeding with the placement of the tissue in an orthotopic or heterotopic site. In this way, the organic tissue 2 can easily be cryopreserved and brought to room temperature only a few hours after placement.

In some non-limiting cases, the device 1 is reusable and the seat 5 can be loaded and loaded again multiple times.

In other non-limiting cases, the device 1 is disposable.

In accordance with a further aspect of the present invention, there is provided an apparatus 40 for the placement of organic tissue 2 comprising at least one device 1 as previously described.

In particular, the apparatus 40 also comprises the portion of organic tissue 2 housed in the seat 5.

In some non-limiting cases, the organic tissue 2 comes from the same patient P who receives it (as indicated above, for example, previously recovered and cryopreserved ovarian tissue) thus carrying out an autotransplant.

In other non-limiting cases, the organic tissue 2 comes from an individual other than the patient P who receives it (for example, whose organic tissue 2 is healthy and compatible), thus carrying out an allograft.

Advantageously but not necessarily, the apparatus further comprises a system 41 for the culture liquid 18, which is configured to inject the liquid 18 from the opening 7 or from an opening that is arranged on the side wall 22 of the base element 3 or of the inner shell 24 as shown in FIG. 3a, through the primary duct 4 and the opening 15, towards the target tissue 8.

According to some non-limiting embodiments, such as those shown in the accompanying figures, the device 1 (i.e. the base element 3) has a rectilinear conformation, in particular along the longitudinal axis A.

According to other non-limiting and non-illustrative embodiments, the device 1 (i.e. the base element 3) has a conformation which is at least partially curved, in particular ergonomic. In such embodiments, the longitudinal axis A is an axis of central symmetry of the primary duct 4, in particular curved. In other words, the course of the axis A follows the curved conformation of device 1.

In particular, in further non-limiting cases, the device 1 (i.e. the base element 3) has a partially rectilinear (on the side of the end 7) and partially curved, in particular ergonomic, conformation in the area of the distal zone DZ, i.e. of the end 6.

In use, the operator O prepares the device 1 (if it is not already ready) by housing in the seat 5 the organic tissue 2 to be implanted, for example ovarian tissue, and possibly culture liquid 18. Subsequently, in the orthotopic/endoscopic site (FIG. 2), the operator O will penetrate the abdominal wall of a patient P until he reaches the retroperitoneal pelvic space 13 (in particular in the area of the ovarian fossa), releasing the tissue 2 below the peritoneum 12; in the heterotopic/percutaneous site (FIG. 1), the operator O will penetrate the skin of a patient until he reaches the subcutaneous tissue 9, releasing there the organic tissue 2. In particular, the organic tissue 2 is released by the operator O by operating the pushing element 14 after he has, if necessary, disengaged the opening 15 from the protection element 16 (i.e. from the obstructing body).

Although the invention described above makes particular reference to very precise embodiment examples, it is not to be considered limited to such embodiment examples, all those variants, modifications or simplifications covered by the accompanying claims, such as for example a different dimension of the device, a different type of organic tissue, a different type of protection element, a different material, etc. falling within its scope.

The device and the apparatus described above have numerous advantages.

Firstly, the invention to overcome the transcutaneous incisional approach (i.e. with surgical incision that allows access to the target tissues) currently in use for the heterotopic reimplantation of ovarian tissue, by using a percutaneous approach.

Furthermore, in the case of an orthotopic approach, the device 1 can be used:
endoscopically/laparoscopically/robotically (i.e. performing, according to the known technique, a surgical operation without performing a laparotomy, but by introducing a camera/optics and surgical instruments such as forceps, scissors, electrocoagulator, suturing machine, needle holders, etc., through small holes made in the abdominal wall), thus allowing to perform only two endoscopic/laparoscopic/robotic accesses (respectively for device 1 and optics) compared to the greater number of accesses (for example four) currently used; or
by video-assisted percutaneous route, thus allowing to perform a single endoscopic/laparoscopic/robotic access for the introduction of the video-optical system (for example at the umbilical level) and realizing the second access directly through the device 1.

In addition, the invention would allow to reduce the time of hospitalization, the invasiveness of the procedure, and would guarantee a better aesthetic result for the patient.

In addition, the organic tissue (for example ovarian) would be damaged to a lesser extent than according to the prior art, as it would suffer less trauma (there is no need to handle it with forceps except for the placement thereof inside the device 1, which is possible with an extremely simple and delicate gesture compared to the placement thereof in the target tissue by means of surgical equipment).

A further advantage of the present invention lies in simplifying the reimplantation of ovarian tissue in patients of childbearing age undergoing cancer treatments at risk of iatrogenic ovarian failure. In particular, the device 1 can be used for the placement/release of endocrine tissues other than ovarian tissue or for reimplantation of ovarian tissue in menopausal patients, so as to prolong hormone support.

Advantageously, compared to the known techniques, the instrument would be atraumatic on the tissue 2 and minimally invasive for the patient (so much so that local anaesthesia is sufficient for the heterotopic approach). In particular, the presence of the protection element 16 allows to prevent the organic tissue 2 from being damaged during penetration, avoiding the entry of fat and other soft tissues into the primary duct.

Finally, compared to the known techniques, in particular in the heterotopic approach, the device 1 can be used individually without the need for additional instruments.

### LIST OF REFERENCE NUMBERS OF THE FIGURES

1 device
2 organic tissue
3 base element
4 primary duct
5 seats
6 first end
7 second end
8 target tissue
9 subcutaneous tissue
10 skin
11 muscle tissue
12 peritoneum
13 retroperitoneal pelvic space
14 pushing element
15 first opening
16 protection element
17 injection device
18 culture liquid
19 inner channel
20 pushing member
21 piston
22 side wall
23 outer shell
24 inner shell
25 second opening
26 cup-shaped body
27 obstructing body
28 end wall
29 retractable element
30 seats
31 mechanical opening device
32 elastic body
33 release system
34 pin
35 membrane
36 flaps
37 portion
38 control system
39 portion
40 apparatus
41 injection system
A longitudinal axis
D diameter
DZ distal area
O operator
OC optical camera
P patient

1. A device for placement of organic tissue, comprising:
a hollow tubular base element (3), comprising, on the inside thereof, a primary duct (4) configured to house, in a seat (5), at least one portion of the organic tissue (2) to be positioned; and the base element (3) comprising a first end (6) and a second end (7) opposite the first end relative to the primary duct (4);
a pushing element (14), configured for insertion on the inside of the base element (3) and coupled to the base element in a sliding manner;
wherein the base element (3) is configured to at least partially penetrate a target tissue (8); and
wherein the base element (3) further comprising a first opening (15), which is arranged in a distal zone (DZ) towards the first end (6) and is configured to let out and position the organic tissue (2) in the target tissue (8) through the action of the pushing element (14).

2. The device (1) according to claim 1, further comprising a protection element (16) for the organic tissue (2), which is configured to protect the organic tissue (2) while the base element (3) penetrates towards the target tissue (8).

3. The device (1) according to claim 1, further comprising at least one injection device (17), which is configured to inject into the target tissue (8) a culture liquid (18), which is configured to enhance the activity of the organic tissue (2) in the target tissue (8).

4. The device (1) according to claim 1, wherein the pushing element (14) comprises a pushing member (20) provided with a piston (21), which at least partially longitudinally goes through the base element (3) and is configured to push the organic tissue (2) through at least the first opening (15) following a relative movement between the piston (21) and the base element (3).

5. The device (1) according to claim 1, wherein the pushing element (14) is a culture liquid (18), which at least partly longitudinally flows through the primary duct (4) and is configured to push the organic tissue (2) through at least the first opening (15) following a relative movement between the culture liquid (18) and the base element (3).

6. The device (1) according to claim 1, wherein the first opening (15) is arranged along a side wall (22) of the base element (3) so as to let out the organic tissue (2) crosswise to a longitudinal axis (A) of the base element (3).

7. The device (1) according to claim 1, wherein the first opening (15) is arranged in the area of the first end (6) so as to let out the organic tissue (2) crosswise or parallel to a longitudinal axis (A) of the base element (3).

8. The device (1) according to claim 1, wherein the base element (3) defines an outer shell (23); wherein the protection element (16) for the organic tissue (2) is at least partially defined by a hollow tubular inner shell (24), which is arranged inside the outer shell (23) in a concentric manner and is configured to house the at least one portion of the organic tissue (2) to be positioned and the pushing element (14); the inner shell (24) comprising a second opening (25), which is available in the distal zone (DZ) and is configured to let out the organic tissue (2) towards the first opening (15); the inner shell (24) being capable of at least partially translating along and/or rotating around a longitudinal axis (A) so as to cause the first opening (15) and the second opening (25) to become complementary to one another, thus determining a connection between the inside of the inner shell (24) and the outside of the outer shell (23) in the target tissue (8).

9. The device (1) according to claim 8, wherein the second shell comprises a cup-shaped body (26), which is configured to contain at least one of the organic material and a culture liquid (18).

10. The device (1) according to claim 1, wherein the primary duct (4) has a diameter which is smaller than or equal to 3 mm.

11. The device (1) according to claim 1, wherein the base element (3) has a diameter which is smaller than 5 mm.

12. The device (1) according to claim 2, wherein the protection element (16) for the organic tissue (2) comprises an obstructing body (27) defined by at least one of:
an end wall (28) of the base element (3); and
a retractable element (29), which is arranged in the area of the first opening (15) and is configured to close the primary duct (4) during the penetration towards the target tissue (8) and to subsequently be retracted into a respective seat (5); and
a mechanical elastic opening device, which is arranged in the area of the first opening (15) and is configured to close the primary duct (4) during the penetration towards the target tissue (8) and to free the first opening (15) by means of a release system (33); and
a membrane (35), which is pre-cut and is configured to be opened by the pushing element (14); and
a portion (37) of the base element (3) in the area of the distal zone (DZ) made of a shape-memory material, which is controlled by a control system (38) so as to shift from the open configuration to the closed configuration; and
a portion of a cup-shaped body (26) defining the seat (5).

13. The device (1) according to claim 1, wherein the seat (5) is adapted to be loaded by an operator with the organic tissue (2).

14. The device (1) according to claim 1, wherein the first end (6) of the base element (3) comprises a cutting profile for at least partially piercing the target tissue (8).

15. An apparatus (40) for the placement of organic tissue (2) comprising a device (1) according to claim 1 and a portion of organic tissue (2) housed in the seat (5).

16. The apparatus (40) according to claim 15, further comprising an injection system (41) for a culture liquid (18), which is configured to inject said culture liquid (18) from the second opening (25), through the primary duct (4) and the first opening (15), towards the target tissue (8).

17. The device (1) according to claim 2, wherein the protection element (16) is configured to assume at least a closed configuration, in which it partially or totally obstructs the positioning of the organic tissue (2) in the target tissue (8).

18. The device (1) according to claim 17, wherein the protection element (16) in the closed configuration at least partially closes the first opening (15); and, in an open configuration, the protection element (16) allows the organic tissue (2) to come out and be positioned in the target tissue (8).

19. The device (1) according to claim 3, wherein the injection device (17) comprises an inner channel (19) on the inside of the primary duct (4), through which the culture liquid (18) flows; the injection device (17) being configured to be operated at least one of before, after or during placement of the organic tissue (2).

20. The device (1) according to claim 6, wherein the first opening (15) is parallel to a longitudinal axis (A).

21. The device (1) according to claim 7, wherein the first opening (15) is defined as a continuation of the primary duct (4).
